(19) United States
(12) Patent Application Publication
(10) Pub. No.: US 2004/0058343 A1
(43) Pub. Date: Mar. 25, 2004

(54) Method of using electromagnetic absorption or perturbation spectra, to diagnose and detect abnormalities in cells, tissues and organisms

(76) Inventor: David M. MacDonald, Irthington (GB)

Correspondence Address: LARSON & TAYLOR, PLC, 1199 NORTH FAIRFAX STREET, SUITE 900, ALEXANDRIA, VA, 22314, US

(21) Appl. No.: 10/350,362

(22) Filed: Jan. 24, 2003

Related U.S. Application Data
(60) Provisional application No. 60/350,295, filed on Jan. 24, 2002.

(51) Int. Cl. C12Q 1/68; G06F 19/00; G01N 33/48; G01N 33/50

(52) U.S. Cl. 435/6; 702/20

(57) ABSTRACT

A method for the detection of normal or abnormal constituents in cells, tissues or organisms, including proteins and nucleic acids, is provided which comprises obtaining a spectrographic waveform pattern of the absorption or perturbation of electromagnetic radiation caused by a nucleic acid, protein, cell, tissue or organism suspected of having an abnormal condition and comparing the pattern with the spectrographic waveform pattern of the absorbance or perturbance of the same radiation by the normal or standard object so as to detect the presence or absence of the abnormality. The method can also be used in a number of diagnostic applications such as sorting of cells by conformational or other differences, such as male from female sperm cells. An apparatus is also provided for carrying out these methods.

### CROSS-REFERENCE TO RELATED APPLICATIONS

This application claims the benefit of U.S. Provisional Application Ser. No. 60/350,295, filed Jan. 24, 2002.

### FIELD OF THE INVENTION

This invention relates in general to a method for utilizing wave mechanical equations and obtaining an absorption or perturbation spectra of a target item, and in particular, to a method and apparatus for measuring the absorption or perturbation spectra from a target item such as a living cell, tissue or organism and using that information to assess properties such as temperature or disease condition of the cell, tissue or organism or the constituents therein. Still other applications within the field of the invention include the development of dynamic models of living organisms for use in the development of drugs and other therapies and non-invasive diagnostics for patient condition and drug templating.

### BACKGROUND OF THE INVENTION

The accurate characterization and understanding of materials and of structures such as cells depends on an understanding of the dynamic nature of the constituent parts. The shape or conformation of the atoms and molecules making up larger entities determines the functionality and performance of both the molecule and of the whole. Conventional characterization relates to static interpretations of the entities and is based on various investigative methods that tend to produce an effect on the entity being observed. Methods such as electron spin resonance investigation, Raman spectroscopy, nuclear magnetic resonance characterizations and electron microscopy and X-ray crystallography observe the behavior of dynamic entities but cannot do so in their functional location. Similarly, separating large molecules using gel electrophoresis and then analyzing them using mass spectrometry characterizes the entities in isolation. Newer techniques of using electromagnetic radiation at wavelengths slightly longer than those of the visible spectrum, in the 10 giga-hertz to 300 giga-hertz ranges, known as tera-hertz (that is 10¹³ cycles per second) have established that each molecular conformation has a particular, unique spectrum, but have equipment and sensitivity drawbacks that the present invention overcomes.

Where the entity is subjected to energy inputs that are significant in relation to the energy state of the observed object, the result may also be affected by the act of observation. At a gross level, the characterization of material properties normally relies on destructive testing and not on comparative methods and the testing of exterior inter-actions with functional entities such as cells can only be carried out in-vivo with limited observations. Where non-invasive technology, such as conventional Raman spectroscopy is used, the depth of observation is normally shallow. It has now been further established that each conformation of a molecule, and each arrangement of atoms, has a distinctive spectrum within the electromagnetic spectrum at a frequency, below that of infra-red emission, in the frequency band ranging from 10¹¹ hertz to 10¹³ hertz, in a region commonly referred to as the tera-hertz frequency bands.

Accordingly, there is a distinct need for a method and apparatus which can utilize electromagnetic radiation so as to obtain a spectrographic absorption or perturbation pattern that can be utilized in the diagnoses and detection of abnormalities in cells, tissues and organs, and which also can be utilized in assessing the ability of drugs or other chemicals to correct certain abnormalities.

### SUMMARY OF THE INVENTION

It is thus an object of the present invention to provide a method and apparatus for using a signal of electromagnetic radiation in the diagnosis and identification of disease conditions and other abnormalities in cells, tissues and living organisms.

It is still further an object of the present invention to provide a method of comparing normal cells with cells suspected of having an abnormality so as to allow early and non-invasive detection of the presence of possible disease or tumors.

It is even further an object of the present invention to provide an efficient and inexpensive method to sense temperature of biological and non-biological materials.

It is yet another object of the invention to utilize wave patterns generated by cells, tissues or organisms in response to a signal of electromagnetic radiation generated as a series of harmonics so as to produce a database of information regarding these biological materials including conformational information concerning ligands and other molecules, viruses and bacteria.

These and other objects are achieved by virtue of the present invention which provides a method and apparatus for the detection of normal or abnormal constituents in cells, tissues or organisms, such as proteins or nucleic acids, which is carried out by applying a signal of electromagnetic radiation to a normal or standard cell, tissue or organism so as to obtain a standard spectrographic pattern of the absorbance or perturbance of the high-frequency radiation by the normal object; applying the same signal to a target cell, tissue or organism suspected of having an abnormality so as to obtain a spectrographic pattern of the absorbance or perturbance of the high-frequency radiation by the target object; and comparing the spectrographic pattern of the normal object with the spectrographic pattern of the target object to determine the presence of absence of an abnormal condition. In addition, methods are provided wherein a spectral absorption or perturbation pattern can be obtained from biological or non-biological materials so as to enable the production of a database containing information concerning the state and condition of the materials.

These and other features of the present invention as set forth in, or will become obvious from, the detailed description of the preferred embodiments provided hereinbelow.

### BRIEF DESCRIPTION OF THE DRAWING FIGURES

FIG. 1 is a schematic view of an apparatus in accordance with the present invention.

FIG. 2 is an engineering view of the components of an apparatus in accordance with the invention.

FIG. 3 is a graphic representation of a spectrographic pattern of healthy tissue versus tumorous tissue as obtained in accordance with the invention.

FIG. 4 is a graphic representation of a spectrographic pattern of a healthy cell profile obtained in accordance with the invention.

FIG. 5 is a graphic representation of a spectrographic pattern of a diseased cell profile obtained in accordance with the invention.

### DETAILED DESCRIPTION OF THE PREFERRED EMBODIMENTS

In accordance with the present invention, there is provided a method for characterizing and differentiating materials such as cells, tissues and organisms, and the constituent materials therein including proteins and nucleic acids, and for constructing in-silico dynamic models of structures. The invention includes the construction of in-silico dynamic models of molecules and arrangements of molecules such that changes in functionality can be modeled and such that the effects of outside electromagnetic influences can be accurately predicted.

The present invention describes collections of atoms as being energy behaving in a way analogous to an eddy or vortex, where the energy is a complex electromagnetic wave. A mathematical non-linear equation describes the wave form (the wave function). The wave function is expressed mathematically as a non-linear equation. Any change in the electromagnetic geometry or three-dimensional sphere of influence of the entity alters the relationship with neighboring entities and as a result alters the functionality of the object. A small change in the geometry has a large potential change in the wave function; the geometry also changes if the energy input to the entity is changed (by for example, heat); that change alters both functionality and wave function description.

The functionality is related to and described by the wave function equation.

Any incident electromagnetic wave is perturbed by interaction with the complex overall wave function of the object. Within the visible light spectrum that perturbation is observed as the Raman effect. It is observed at radio frequencies immediately below the frequencies of visible light in an area referred to as terahertz frequencies, being frequencies of 10¹³ hertz. Thus, if the incident wave is made up of an equally balanced spectrum of wave lengths, and one or more of those wavelengths is then perturbed by inter-action with the object, the result of the perturbation is a shift in frequency that can be observed.

At longer wavelengths, the same shifts occur, but have not been so readily observed. The advantages of using longer wave length methods than are employed in Raman spectroscopy are that in situ and in vivo interrogation is possible. The depth of penetration of an electromagnetic wave is proportional to its wave length, for a given intensity.

The present invention compares changes in the amplitude of the various component wave lengths of the signal and by so doing determines the frequency shifts. Observing the incident wave in the absence of an object establishes the spectrum of the received signal. The incident wave is now used to inter-act with the object wave function and the amplitudes of the various components of the resultant wave function are observed and measured. Comparison of the signal in the absence of the object with the signal produced by inter-action with the object produces the unique spectrum for the object.

If a coherent or continuous-wave electromagnetic signal is transmitted, that continuous wave signal is perturbed in a unique way by incidence with specific molecular conformations. Examples are in the use of tera-hertz transmissions to identify both qualitatively and quantitatively particular molecules, or particular conformations. Similarly, recognizing the spectrum of the metabolite, using an NMR scanner, can identify the presence of specific metabolites. The use of differential staining (where changes in conformation can be recognized by their different perturbation of visible light and where the staining usually with a fluorescing dye emphasizes the frequency shift) is part of the same phenomena.

Experimenters have tested this theory by trying to produce stable high frequency continuous wave signals to measure the resulting perturbation, but the production of stable high frequency signals (in the near infra-red spectrum) and scanning down the frequencies, has previously shown to be extremely difficult and to require very complex and expensive equipment.

At high frequencies (that is, just below the infra-red frequencies) there is still a problem with diffraction and more particularly since the frequency of the molecular vibration is in the same frequencies, there are effects of temperature. In other words, if the vibrational frequency changes (perceived as heat since the emission is in the infra-red frequency) the conformational change identification is also made more complex.

The technique of tera-hertz time-domain spectroscopy makes use of the coherent detection of broadband pulses to analyze or identify the spectral response of materials.

The interaction of high frequency electromagnetic radiation with a specific object is expressed in the relationship between the frequency dependent refractive index of the object, which is conformation as well as constituent dependent, and the degree to which a specific frequency is absorbed by the material. At visible light wavelengths, the relationship is referred to as the Raman shift.

The relationship can be written as:

N(v)=n(v)+c(frequency specific absorption co-efficient/4pv, where N(v) is the complex refractive index for a frequency, n(v) is the real refractive frequency dependent index, c is the speed of light and v is the incident frequency.

The production of high frequency coherent signals in the tera-hertz frequency range is difficult: their detection and analysis is complex and the signal to noise ratios make accuracy difficult. Complex equipment is needed and, since the depth of penetration of any electromagnetic radiation is proportional to the wave-length, with long wave lengths having the greatest penetration in vice versa, a signal created and transmitted at near infra-red frequencies has limited penetration.

The present invention is that if a lower frequency coherent or continuous wave signal is created and the higher frequency harmonics established mathematically (using a Fourier transform) any interaction at high frequency (if analyzed using the same Fourier transform), will be seen as a specific spectrum. In other words, by going up the frequency range from a relatively simple coherent or continuous wave signal at a lower frequency, the conformational differences, in spectral terms, can be recognized quite easily. Any transmitted radiation, at a wavelength that allows penetration of the subject, will contain the principle wave length together with all harmonics of that wave length.

Thus, a longer wave length signal that is penetrative, contains within it shorter wave lengths. Those shorter wave lengths are perturbed by interaction with materials in a specific way and that perturbation occurs in all harmonically related wave-lengths. The perturbation produces a spectrum.

That spectrum is unique to the nature and characteristics of the object. A change in the conformation of the molecular constituents of the object, which change induces a functional change in the object as an entity, is exhibited as a change in the spectrum. Changes in the vibrational frequencies of the molecular constituents of the object, commonly observed as a change in temperature, result in a change in constituents of the spectrum.

Where objects that are identical in terms of their particle components but of different functionality and therefore of different conformation are compared with this technique, a means of differentiating materials of different functionality and of characterizing the energy difference between the two functions is therefore available.

For example, various proteins present in the brain of mammals exist in different conformations or geometries, but with identical chemical analysis. Examples are ameyloid precursor protein, tau protein and prion protein. In each case, the protein exists in a normal conformation and in an abnormal conformation and the latter has significantly different functional effects.

Experimental work has established that each different conformation of a molecule has a particular absorption spectrum in the high frequency range. The corollary of that is that the perturbation spectra is also different: the perturbation spectra is different from, but related to, the absorption spectra. The absorption spectra for a specific conformation is made up of the constituent part absorption spectra, together with the absorption spectra for the system as a whole. The present invention describes the application of a broad band signal, generated as a series of harmonics based on a lower frequency principal signal, to an entity and the comparison of the perturbation of that signal with an unperturbed signal. The result of this approach is that specific entities can be readily identified even when surrounded by similar entities of different conformation, or where surrounded by the constituent elements of the target.

The di-electric characteristics (dielectric constant, relative permittivity and loss tangent) of materials and tissues have been observed and recorded, typically using equipment that comprises a signal generator and a network analyzer. The effect that is measured is the -apparent absorption of electromagnetic energy by the subject material. Some high frequency spectra have been recorded using near infra-red transmissions linked to an optical or o0ther detector.

For the practical use of the present invention a database that identifies the various wavelength shifts of specific objects and of specific functionalities is desirable. The apparatus for such observations requires to be commercially available so that any observations that are made are not affected by differences in the signal generator or processing hardware.

A typical arrangement therefore consists of:

1. A wide bandwidth signal generator, able to generate a signal in the frequency range from 100 mHz to 2 GHz

2. A transmitting antenna and an identical receiving antenna

3. A suitable low noise operational amplifier

4. A wide bandwidth oscilloscope equipped with appropriate software to allow the signal to be analyzed.

In general, commercial items for function 1 include Rf generators such as those made by Thurlby Thander, Tektronix, Fluke and others. Typically, these units include the ability to produce pulsed or continuous wave form signals and incorporate significant levels of signal processing.

The transmitting and receiving antenna can range from simple wave-guide forms such as co-axial cable terminations through maser-like structures to the bow tie antenna designed by NASA. Maser tube antenna, filled with a high dielectric are convenient in that the physical dimensions for a simple dipole with a primary transmitted frequency of 100 MHz are reduced by the comparative dielectric of the tube filling. In all cases, the production of a coherent signal is desirable and therefore the aperture should be kept as small as is possible. The two antennas should be identical to provide a balanced signal.

The provision of a very small aperture to encourage the production of a fine diameter coherent collimated signal is desirable. The effect is similar to the optical effects of a pinhole camera. Apertures of less than 20 microns in diameter are desirable in maser tube applications.

The low noise amplifier is required to produce a sufficiently large signal to allow the oscilloscope to analyze the signal.

Wide bandwidth oscilloscopes are commercially available (typically the Tektronix 7000 series, for example) incorporating processing software.

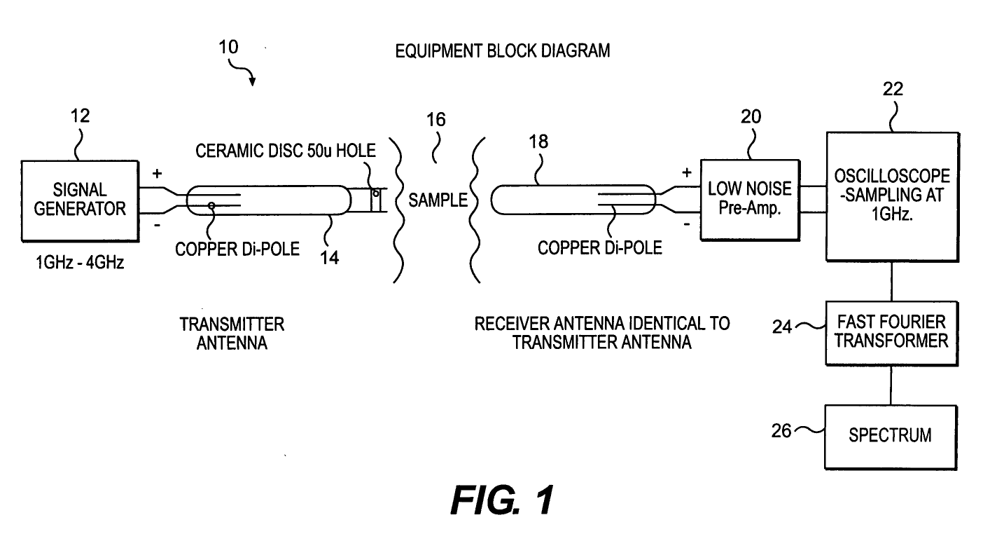

An example of an apparatus of the invention is shown in the schematic drawing of FIG. 1. In this drawing, the apparatus 10 comprises signal generator 12, preferably one which can transmit a signal of electromagnetic radiation, e.g. at a frequency of between about 1 and 4 gigahertz, a transmitting antenna 14, which in the embodiment shown is a copper dipole antenna having a ceramic disc with an opening of 50 μm, and an area 16 for retaining a sample between the transmitting and receiving antenna. Next, the apparatus includes a receiving antenna 18, which in this case is a copper dipole antenna. The receiving antenna 18 is connected to a suitable recording or analyzing device such as oscilloscope 22, and in the preferred embodiment, a low noise pre-amplifier 20 will receive the signal from antenna 18 before it is received at oscilloscope 22. The information regarding the electromagnetic signal received at oscilloscope 22 is then transmitted to a suitable transforming function such as accomplished by a Fourier transformer, in this case Fast Fourier transformer 24, and through the Fourier transformation, and a spectrographic waveform pattern or spectrum 26 is created by the transmission of the electromagnetic signal through the sample which is used to assess the properties of the materials tested, as explained in more detail herein.

The procedure for establishing base data of the frequency/wave length shifts is to calibrate the apparatus, without the object being in place. The configuration can be either where the object is placed in the direct beam between transmitting and receiving antenna or where the transmitted signal is reflected back to the receiving antenna. The spectrum of the received signal can be derived by expanding the wave form of the received signal using a suitable mathematical expansion such as a fast Fourier transform, which is conveniently incorporated in the oscilloscope.

The system is comparative. If no object is present, the received signal is expanded by a time domain system to synthesize the harmonics of the signal. The object to be characterized is then introduced and the resulting received signal is then compared with the unperturbed signal and a shift spectrum established by a process of interferometry.

The disturbance caused by the electromagnetic characteristic of the object may be out with the actual transmitted and received frequencies. However, a disturbance at a higher harmonic has an effect at a lower harmonic of the same signal and provided that the same mathematical method of synthesizing the harmonics as used, the disturbance at high frequencies affects the lower frequencies and can be synthesized using the time domain expansion.

Where the system is to be used to diagnose or identify, this process produces a shift spectrum where specific wave lengths are shifted by the wave function interaction and as such are identified by changes in the amplitude of the received signal at the wave lengths that are shifted. Where a wavelength is shifted from, a reduction in the amplitude of the received signal compared with the amplitude of the same wavelength in the unperturbed signal is observed. Where a wavelength is shifted to, an increase in its amplitude is observed.

The same techniques can be used to characterize differences in function of otherwise similar objects. For example, microbially produced manganese dioxide differs from synthetically produced manganese dioxide. That difference relates solely to the electromagnetic geometry of the molecule.

The difference can be characterized. The changed functionality of a larger entity can also be characterized. Where, for example, a cellular organism is observed, the overall wave function of that organism perturbs the incident wave. Changes in the electromagnetic geometry of the constituent molecules of the cell result in changes in the functionality of the organism. Conversely, a change in function imposed by an outside event results in a change in the electromagnetic geometries of the molecules constituting the cell. In either event, some of those changes are pronounced. The overall effect is to alter the wave function of the entity as a whole and therefore to alter the spectrum of wavelength shifts that characterized the cell in its first functionality.

The spectral characteristic is specific to the vibrational frequency of the entity: that frequency is observed as temperature and as a result, the invention can be used to identify temperature of an object very accurately. Where a larger entity is observed, such as a living organism, the functional change results in metabolic changes that in turn have small but identifiable spectral effects.

The wave function of any entity describes that entity. Therefore, the interaction of a wave function with the wave function of an entity is singular.

A complete database of the wavelength shifts of potential target entities can be established. Once the wavelength shift spectrum has been established, simpler apparatus can be used for diagnostic or test purposes.

That apparatus consists of a circuit producing a signal comprising the various principal wavelengths that radiation is shifted from and a receiver observing the wavelengths shifted to. The level of energy received identifies the presence or absence of the target entity or functionality.

Where a database of the spectral difference between functions has been established, the difference can be described by a non-linear equation comprising quanta and frequency of energy. That equation describes the amount and form of energy utilized in bringing about the geometry changes that result in the functional changes.

It therefore provides a useful indicator of the means of reversing such a change and in the instance of a dysfunctional organism can indicate a suitable therapy to reverse the change. By matching the negative of the non-linear equation that describes the energetic changes between differing functionalities with the characteristics of a compound, by example, suitable potential drug candidates to reverse the functional change can be identified.

It is accordingly of significant importance that organisms at the initial point of functional change are examined rather than organisms where the primary functional change has been multiplied by the consequences of the original primary functional changes themselves. If a functional change increases the rate of ion transfer into a cell, for example, the effects of that increase are themselves to bring about conformational changes in the proteins of the cell and, eventually, to bring about more fundamental changes.

Specific applications include, by example, the ability to differentiate between healthy neurons and neurons suffering from degenerative diseases, where the degeneration results in a diminished function and a diminished metabolic rate. Similarly, a cell developing symptoms of cancer will exhibit increased metabolic rates, characterized by increased emission with the infra-red spectrum and associated changes related to calcium ion transfers. In both of these examples, the invention allows for early in vivo diagnosis.

In another application, the present method can be utilized to determine the presence of abnormal prion proteins, such as are responsible for “mad cow” disease.

A method is described whereby the presence or absence of a specific conformation of a protein can be identified in a living organism or animal, including a human, rapidly and non-invasively. An example is the prion protein associated with the transmittable spongiform diseases of the brain, for instance BSE in cattle: the PrPsc form of the prion protein is a different conformation of the normal prion protein produced by neurons.

The identifying spectrum for normal prion protein and for the unusual conformation prion proteins is first established by exposing samples of each material to a coherent wave signal as described herein and determining the [perturbed signal resulting from the interaction of the transmitted signal with the target material. That spectrum can be determined irrespective of whether the target material is in solution with other materials.

Where a specific spectrum is detected associated with the conformational abnormality of a protein linked to a disease, the method is used in the templating of drug candidates for therapies intended to correct the conformational abnormality. The spectral difference observed as described herein can be quantified in terms of frequency and amplitude: that is done by subtracting the spectrum of the normal conformation from that of the abnormal. The difference between the two, expressed in frequency and amplitude, is the energetic characteristic difference (ECD). The ECD is net of the energy required to initiate the change (to overcome the inertia of the normal conformation). The ECD can be written as a non-linear wave equation that describes the difference: the opposite of the ECD (that is, by substituting negatives for positives and vice versa) is an indication of the energy, net of the energy required to initiate change, required to reverse the change. The system thereby provides a useful indication of candidate drug compounds or therapies that can result in the conformational change being reversed and the resulting cell abnormality being corrected.

Accordingly, a method is provided wherein drug templating of candidates for particular drug therapies is carried out comprising identifying a specific spectrum of a protein associated with a conformational abnormality linked with a disease by scanning said protein with a signal of electromagnetic radiation so as to obtain a spectrographic absorption or perturbation pattern of said conformational abnormality, introducing a drug suspected of treating the disease to the abnormal protein and obtaining an electromagnetic scan of the protein after interaction with the drug, and determining the efficacy of said drug by comparing the conformation of the protein following treatment to determine if the conformational abnormality has been reversed in whole or part.

In general, for mass in-vivo examination, an apparatus consists of the method of producing a coherent principal signal as described herein together with the receiver and pre-amplifier described. A detector circuit can be used to determine if the specific spectrum associated with the target material is present. By scanning the candidate the presence or absence of that specific spectrum is found. Such a scan is very rapid: provided that a number of pulses of the transmitted signal are made the probability of the target molecules being absent or present, depending on whether the specific spectrum is observed, can be very high.

The scanning device can most conveniently be a simple probe, with a head containing both transmitter and receiver antenna. The head can be passed closely over the target area.

The method is intended primarily for the detection of conformational differences in proteins present normally where those conformational differences result in cell degradation diseases. It is anticipated that the method will prove to be specifically useful in the identification of early stage protein changes related to for instance cancer as well as the degenerative diseases of neurons described above.

The method can also determine the presence or absence of specific molecules in small quantities and is useful therefore in determining whether narcotics are present, or to determine the presence of metabolites specifically associated with cardiac disease.

The changing characteristics of sperm cells as they mature appears to indicate that initially male and female sperm cells are at different points in their functional change and that functional change is characterized by alterations in the ion transfer rates. As a result, the invention allows the proportion of male to female cells to be determined and the cells can then be divided into male and female groups.

In another specific embodiment of the invention, a cell sorter is provided which makes use of the method of the invention. A method to sort cells by bulk iterative methods. Where cells of two or more types are mixed together, such as in the ejaculate of a male mammal, the present invention can be utilized to sort the cells. The spectral difference between, for example, a male cell and a female cell is established. A sample already sorted into cells of one type is scanned by passing a coherent wave through the sample and recording the perturbed signal. That spectrum is compared to a similar signal obtained by scanning the cells of the second type. Comparing the two spectra the critical differences within the spectra, in terms of frequencies are established. One or more specific principal frequencies can be selected: in the case of cells of one sort, the perturbation results in a significant frequency shift from a principal frequency: that shift is shown as a decrease in the amplitude of the transmitted frequency and an increase in another, adjacent frequency. In the case of the cells of the second type, a second principal frequency with its commensurate shifts is selected.

Where a mixture containing quantities of the two types of cell is scanned with a coherent signal containing the two principal frequencies described, the relative amplitudes of the shifts can be measured. By applying the rule of mixtures, the relative amplitudes indicate the proportions of the two cells, each to the other.

In accordance with the invention, a method for differentiating and separating male and female sperm cells is provided which comprises obtaining an animal ejaculate and dividing the ejaculate into portions to be scanned with electromagnetic radiation; scanning said portions of the ejaculate with electromagnetic radiation and determining the percentage of male and female sperm cells in the scanned portion by comparing the spectrographic pattern of the absorbance or perturbance of the radiation by the portion with the known spectrographic patterns made from an ejaculate having equal numbers of male and female sperm cells; separating those portions which have a predominant amount of male or female sperm cells; and repeating the above steps and collecting the ejaculate portions containing sperm cells of predominantly one sex until a desired percentage of sperm cells of that sex is obtained.

One embodiment wherein a machine is utilized to carry out the present invention is described below:

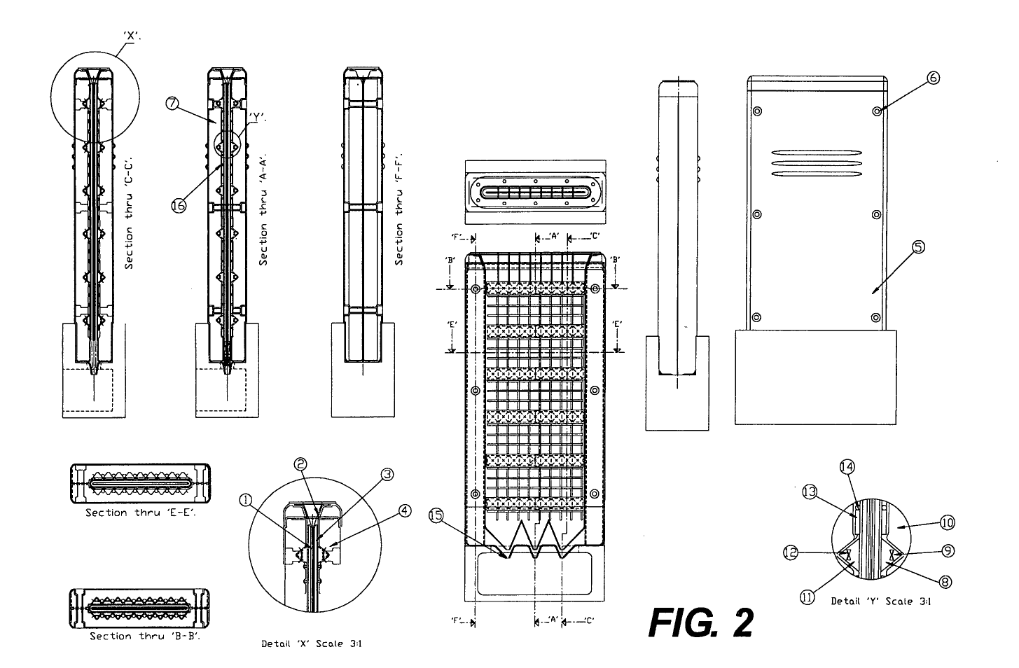

The machine is intended to sort animal semen into male and female, with a high probability (in excess of 93%) and a residue of indeterminate probability. The method of operation is to load the machine with untreated ejaculate, through a load funnel. This produces nine equal portions, each of which is held in a determinant area. Each determinate area is faced with an RF transmitter and backed by an RF receiver in accordance with the invention. When the area is subjected to a pulse of specific wavelength, the absorption of energy is measured by comparing the transmitted energy, less the known normal loss of the system without ejaculate, with the received energy. A depiction of the elements utilized in an appropriate cell sorting system in accordance with the invention is shown in FIG. 2.

The absorption at that frequency of male cells and of female cells is known and by applying the rule of mixtures, the proportions of male and female cells can be determined. Samples that are predominantly male (60% or higher) are then sorted to one side and redivided. Samples that are predominantly female are sorted to the other side and redivided. The process is then repeated. Where a sample exhibits a high proportion of one sex or the other it is moved toward the end of the sort process and stored there until further samples of a similar concentration are derived. The methods of moving the ejaculate mimic the natural peristaltic action of ejaculation. The sample is kept between two, thin, flexible membranes. A layer of magneto-rheological fluid that increases steeply in viscosity and density when a magnetic field is applied to the backs of each membrane. By applying localized magnetic fields to the fluid, the space between the membranes can be minimized and the sample moved or stored at will.

The general components of the sperm cell sorting system of the present invention are as follows. The cell sorting machine used in the process described above has main components including an outer casing, clamping mechanism, entry funnel, membrane glove, magneto-rheological fluid, encapsulated sensors/fields, an exit funnel and an electronic control unit. The device may be manufactured in any suitable way, and in one specific process, the outer casing is manufactured in two, opposing halves made from a suitable thermoformed plastic (e.g., ABS, high density polyethylene, etc.), but can if desired be gravity die cast in aluminum. The benefits of ABS moulding are reduced part price, reduced tooling cost, reduced time to production and good but limited protection from impact damage. The two halves locate to each other using dowel pins with butterfly nuts at each end. The dowel pins locate in sleeves hot-staked into the moulding. (If an aluminum casting is used, the sleeve is a drill and ream operation.) The casing is molded with vents to allow the electronics to be cooled and incorporates a single DIN socket on one half and two DIN sockets on the other. One DIN socket carries the ribbon cable connector between the units. The second is the power supply and data link. The casings are provided with a means to be stand alone units but also have a carrying strap connection that allows the unit to be hung on, for example, a stand.

The clamping device fits over the exterior halves at the top and positions the casing correctly. The membrane glove is drawn up though the center slot and clamped in position by the dividing funnel. In operation the ejaculate is poured into the funnel and the division divide the ejaculate in the nine samples. The membrane glove is, in effect, one glove sealed inside another, with a flange around the edge that is trapped within the outer casing. The space between the inner and outer glove is filled with magneto-rheological fluid (such as that manufactured by Lord Industries). The glove is made from soft nitrile or latex material and the inner and outer are heat staked together after filling with the m-r fluid to contain the fluid evenly in the interstice. The glove is also heat staked so that it is divided into vertical sections in the antenna areas. The magneto-rheological fluid may be any suitable such fluid, for example, the fluid manufactured by Lord industries. It is now being used in adaptive damping systems for General Motors and is therefore widely and economically available. When excited by a local magnetic field, the fluid becomes very viscous (close to solid) and as a result the localized volumes increase. The response rate for the change is in the order of milli-seconds.

In summary, a suitable apparatus for carrying out sorting of sperm cells by sex includes a means of generating and receiving an electromagnetic signal such as a broad band signal with wave lengths between 1,000,000 and 1 meters; an antenna for directionally transmitting the signal and an antenna for directionally receiving the signal; a means for receiving and portioning an animal ejaculate; a means of calculating the absorption pattern of electromagnetic signals after said signals are directed to portions of the ejaculate so as to determine if said portions contain a predominant percentage of sperm cells of one sex; and a means for collecting portions of the ejaculate which are identified as containing sperm cells which are predominantly one sex at a desired percentage.

Other important elements for this device include the antennae and the electromagnets that generate the magnetic fields. The antennas are preferably pyramidical in shape and are formed by vacuum forming a polyester (Mylar) film of a thickness of between 100 and 150 micron. The vacuum formed pyramid shapes are part of a larger vacuum forming that encompasses 3 mm diameter hemispherical slots arranged at 0 degrees and 90 degrees to the X axis of the machine. Each slot can carry an electromagnet that consists of a fine wire winding over a polymer core. The outward facing (that is, facing towards the sample) face of the Mylar vacuum forming is metallized. A simple vacuum formed pyramid is metallized with a ‘bow tie’ dipole at 0 degrees to X, with a second identical part metallized at 90 degrees to X. One such forming is inserted in the main pyramid to form respectively the transmitter and the receiver. The dipole has a wire termination that is inserted through small holes in the main pyramid; these holes are formed during the vacuum forming process. In this way, the spacing between the reflector and the dipole is maintained. Following assembly and insertion of the electromagnets, the entire assembly is encapsulated using a standard potting compound, to provide a smooth and flat surface against which the glove is stretched. The exit funnel for the glove allows for three drains as described and stretches the base of the glove into position.

In this apparatus, the electronic controls consist of a printed circuit board that backs the encapsulated parts (F). The circuit board provides discrete connections to each transmitter and to each electromagnet. The system is driven using standard algorithms with the e.c.u. On initiation, a series of 100 pulses is transmitted from each of the first nine antennas in turn and the received signal analyzed. The received signal is compared against the received control signal that is established by transmitting with no ejaculate in the system. The result is then compared against the established absorption levels for male and female cells. The absorption levels indicate the proportion of male to female cells. At that point, each antenna pocket is given a value derived from the male: female absorption. The ejaculate is confined laterally by the heat staking and vertically because the electro-magnet at the base of the antenna pocket is activated, so that the expanded magneto-rheological fluid forms a barrier. The samples are now moved vertically and laterally using the magneto Theological effect so that nine new division can be created graded as to the highest male in the one or two left hand antenna pockets, the highest female in the one or two right hand antenna pockets and the balance equally redivided across the center pockets. The process continues until at least three antenna pockets on the left have an acceptably high male proportion and the three on the right have an acceptably high female proportion. Because the sample can be moved vertically up and down between antenna rows, the first stage sort/measure process can be repeated until an improvement over the first sort has been obtained.

With regard to software, the information aspect of the sorting device preferably comprises a pulsed transmitter and a filtered receiver. The machine can be supplied with alternate frequencies so that the same device can be used for different species semen or alternatively to predominate male or female cells. The receiver filter can also be set to different values. Since the machine is intended to self calibrate (that is, the background noise and absorptions are zeroed by cycling the machine dry, without ejaculate) these adjustments can be carried out within software by pulsing consecutively at rising frequencies and choosing only to analyze the received pulses in a sequence that relates to the sample. In other words, where the machine is set to be able to handle five different species, it will pulse a sequence of five signals. The receiver will only read every fifth signal, commencing with the one appropriate for the sample under consideration. It is intended that a complete sort record will also be maintained and it is possible that the final stage of each sort can give some degree of quality control by being programmed to consider a protein conformation.

The ability to characterize wave function changes has utility in applications other than those concerned with living organisms. These applications are widespread in manufacturing science, providing convenient means of monitoring and controlling quality.

For example, measuring the rate of ion transfer through a material can indicate the degree of orientation and the strength of linkages in polymers. Since the ion transfer rate is determined by the electromagnetic geometry of the entities making up the material, the present invention provides a convenient way of measuring these changes. By confining such examination to long wavelength radiation, the results can be provided throughout the object. Such examination provides a comparative measure of the toughness of the object. Where a comparative measure of the strength of a material is needed, the behavior of entities under load can be used.

Where a load is applied, that load is experienced at atomic or molecular level as changes in the electromagnetic geometry of the entities and as such the perturbation of the incident wave is related to the behavior of the entities as load is applied. The results can be compared with the database derived by examination of a known sample whose behavior can then be determined by destructive testing.

In the case of remote temperature measurement an interferometry spectra can be obtained for each significant change in the vibrational frequency of the object. By comparing the experimentally derived interferometry spectra with known energy inputs, the inertia levels of the entity can be calculated. The method provides a useful measurement for metabolic changes in living organisms as well as for industrial applications. In the case of living organisms the metabolic change of a specific arrangement of entities (a specific cell type) can be monitored even where that cell is only present in certain tissues. It is likely that the core metabolism of a living creature can be monitored and comparison with surface metabolisms provides a critically important indication of system function.

Similarly, it is possible to monitor changes, either caused by external heating or caused by internal exo or endo-thermic reactions, in the mass of inert materials.

Still other applications of the present invention are possible.

The production of a proteome database (especially human) is now a crucial development in biology, but the production of that database is a hugely complex undertaking compared with the production of genomic databases, especially human.

By regarding the cell as a dynamic energetic system where all of the constituent components are inter-related, a different and novel approach to identifying, defining and characterizing the proteome may be adopted.

The basic ingredients of the cell are known: the amino acids, water, trace elements, sugars etc and these constituents can be characterized by their non-linear wave functions. These basic elements are dynamically stable.

Treating the cell as a series of wave functions, with the nucleic acids providing a stable electromagnetic base description, solutions can be proposed until a solution that fits the functional experience of a simple cell is produced. Describing an entity by a wave function describes its energy: any entity is constantly gaining and losing energy and its description fluctuates. If energy is added at a greater rate, or lost at a greater rate, than is the norm, the fluctuations are more or less extreme. These changes in conformation are recognized as being changes in the temperature of the entity and are shown by changes in the wave function of the material.

The present invention proposes that the wave functions of a cell be reproduced as a mathematical model and the relationship between the wave functions be governed by the laws of physics that determine the turbulence of any stream flow.

The analogy is to the modeling of stream flows. The nucleic acids provide the streambed. The energy inputs are the metabolic rates of the cell. The protein conformations are the vortices that form in turbulent stream flow.

Each vortex can be described by a Schrodinger equation and that equation may equate to a three dimensional description of a protein molecule.

Practically, to create the proteome by analysis is a lengthy and inaccurate process. Protein molecules are highly dynamic and the only information that external analysis provides with any certainty is that the ex vivo conformation is NOT the same as the in vivo conformation. Thus conclusions drawn from an ex vivo examination and discussions of the function of a protein based on ex vivo examination is likely to be misleading.

The alternative is to apply established rules of basic physics and to build what amounts to a self-assembly model. A single, unique solution exists for any given energy parameter (metabolic rate) in a given environmental arrangement, with a specific DNA and with specified initial volumes of constituent elements.

That solution will depict molecules (including proteins) as wave function equations. Those wave functions are the mathematical descriptions of three dimensional depictions of the molecules and describe the dynamics of the three dimensional molecule.

The dynamic model depends on the graphing of complex non-linear equations (typically Schrodinger and Wheeler Feynmann equations, as opposed to Navier Stokes equations) and the application of semi Lagrangian algorithms to produce partial solutions that can be graphed.

The solutions are extremely complex and a method to solve the various relationships is needed that is faster than iterative solutions. Parallel processing systems, generic or agent based programming provide suitably accelerated methods.

Fundamental physical laws govern the behavior of adjacent wave functions. Those laws relate to the interaction of all turbulent flows of any sort. Turbulent flow is determined by the fixed surroundings of the stream. The present invention describes a method to model the dynamic structures of living organisms by using the fixed electromagnetic geometry of the nucleus molecules, typically deoxyribonucleic acid, as the equivalent of a stream bed and the metabolic rate of the cell as the equivalent of stream flow, to produce the singular turbulent flow pattern related to the interaction of the wave functions of the constituent particles.

In the model, the vortex and turbulent eddy sizes and complexities are representational of the electromagnetic geometry of the object. Large established vortices represent macro-molecules and smaller persistent turbulence represents the presence of simpler molecules.

As in any stream flow pattern, for a given flow rate, given flow densities and for a fixed environment, there is only a single solution comprising the constituents of turbulent flow. Any alteration of any component, by any external input, has a series of consequential effects, changing the geometry of the vortices and eddies. In a dynamic model the small entity representations have high resistance (inertia) to change and the vortices have lower inertias, representing the likelihood of conformational change. In turn the conformational changes result in flow changes, where the flow represents the metabolism of the entity.

Significant changes can cause highly energetic turbulence and as in a stream model, such flow can alter the previously fixed environment, either by altering the geometry of the nuclear conjugations and in turn altering their collective geometry or by altering the electromagnetic geometry of the nuclear constituents.

The mathematics and logic are related closely to magneto hydro dynamic modeling utilized in astrophysics, as well as relationships with certain non-linear flow dynamics models.

A novel method to identify, describe and measure both quantitatively and qualitatively the constituent components of a cell, tissue or organism and to characterize and predict the dynamics of the target cell, organism or tissue has been devised.

The very large molecules (proteins) that make up the constituent parts of living cells are extremely energetic. As such they disperse energy at a significant rate, perceived normally as heat or temperature, and in order to survive they absorb energy.

When such an entity is examined ex-vivo (for example, separated by gel electrophoresis and examined by mass spectrometry), its dynamics are changed radically. In order for the entity to survive to be studied, the vibrational frequencies are massively reduced and therefore the dynamic structure is radically altered.

Thus, any ex-vivo examination may be less than accurate and could prove to be misleading.

The linear polymers (the nucleic acids) appear to be far less energetic and although the conformation of nucleic acid molecules may be significantly different in vivo than ex vivo, the dynamics are of a lower order than the dynamics of protein molecules.

The electromagnetic environment surrounding the molecule influences the wave system of the molecule. If localized high energy exists that localized effect on the overall wave system of the molecule will affect the rest of the wave system and by extension will have similar but reducing effects throughout the environment surrounding the molecule.

To all practical effects that change is instantaneous and can be considerable.

Since the wave velocities are fixed (at c), then one can equate energy density with velocity in a conventional stream flow. By doing this, a representation of not only a molecule but an arrangement of molecules within an environment can be created using computational flow dynamics. That model would also represent energy loss and energy replacement. In the case of a cell, the complex inter-action whereby the fact of energy transfer into the cell by way of, for example, ion pumping affects not only the metabolism of the cell but where the ion density of the cytosol determines the geometries of the protein molecules in the membrane and therefore in turn regulates the ion transfer rate could be modeled. Such a model would allow an examination of the effects of localized energy inputs, at differing wave function characteristics.

In order to determine the parameters for the model, a wave function for either a molecule or an arrangement of molecules has to be postulated.

To do so empirically is an imposing and probably impossible task.

However, the wave function of a target will perturb an incident wave. The wave function of the incident wave is known, as is the wave function of the resultant wave after interaction with the target wave function.

By varying the functionality of the target (for example, between crystalline and amorphous forms) the change in geometry can be expressed, again in a wave function equation. By allying that with fundamental knowledge of, for example, changes in inter-molecular performance, a model can be constructed.

If known effects then change the target material and the physical results of those changes are observed, a comprehensive set of data representing the dynamic activity can be assembled.

Since the wave functions of simple atoms can be determined relatively easily, a series of rules of simple atom interaction can be tried and a gradually more comprehensive model built. It is probable that the model only needs to know the rules governing the wave function of the outer layer of a molecule.

It has been established that any atom or collection of atoms has a particular spectrum of dielectric properties at specific frequencies of the electromagnetic spectrum. These can be identified at radio frequencies in the range between 50 MHz and 1 GHz and are expressed as being permittivity, dielectric constant or loss tangent.

The work has been published by amongst others the Brooks Air Force Base unit of the United States Air Force department of Aerospace Medicine. This examination of dielectric properties identifies the radio frequencies where materials or cells are most likely to absorb energy.

However, the dielectric constants change with alterations in the geometry or conformity of the target and therefore by comparing the loss tangent/frequency spectrum of different conformations of the same material, valid conclusions can be drawn as to the energy input required to promote the conformational change.

It is known that a point field charge will promote a conformational change in proteins. That conformational change will bring about functional changes in the mechanism the protein is part of. The overall difference in function results in a different energy signature and therefore allows identification of the presence or absence of dysfunctional systems. It also allows some conclusions to be drawn concerning the energy input.

The shape of a cell whose plasma membrane is made up of proteins, ligands and other molecules is integral to the function of the cell. The distribution of charge over the surface of a cell affects the conformation of proteins within the plasma membrane and the charge distribution may alter the shape of the cell itself. In either case, a gross spectrum characterizing the cell shape can be observed, and the conformational spectrum of specific, identifiable constituent entities of the cell can also be determined. By determining the presence or absence of entities of a particular conformation, early stage diagnosis of cell changes can be made. By establishing the difference between conformations of the same entity in energy terms, an indication of the energy profile required to bring about, or to reverse, the conformational change can be predicted. That energy profile can then be matched to the energy profile (wave function description) of the active constituent of a drug.

The loss tangent/frequency relationship is governed by the wave function of the target: the wave function is derived from the numbers of electron shells, the electron density and the other quantum mechanical characteristics of the molecule. A key aspect is the change in rotational frequency produced by energy input: what is normally referred to as heating.

However, since the proposed technique is comparative, in most cases the changes due to heat energy input are irrelevant.

The wave function of the illuminating signal is known, as is the wave function of the perturbed or altered signal. The difference relates to the loss tangent.

That difference can be expressed as a wave function: the resultant of the interaction between the wave function of the signal and the wave function of the target produces the wave function of the output. By comparing the two wave functions resulting from the different functional states of the target, a wave function of the energy signature of the changed state can be derived.

Based on the understanding of molecular dynamics here set out, a number of applications are contemplated.

1. Through the development of a database of spectral differences between similar functions, apparatus to identify the presence or absence of specific functions is possible.

2. The database is developed by plotting the frequency shift at electromagnetic wavelengths longer than those of visible light. By comparing the frequency shift of chemically similar conjugations that exhibit differing functionalities, specific electromagnetic frequencies can be identified at which there is a measurable frequency shift.

3. That frequency shift (identical to that characterized as the Raman effect at visible light wave lengths) is recognized at lower frequencies by an apparent energy absorption at that specific frequency. [If a frequency shift is present, that frequency shift results in the amplitude at the parent or original frequency being reduced and the amplitude of the frequency to which energy is shifted being increased]. The frequency shift (that is, the principal frequencies that are modified and the amount by which each principal frequency is modified) is entirely particular to a particular substance, molecule or collection of molecules.

4. It is important that the wave characteristics of the target are non-linear. The interaction of non-linear systems gives rise to more substantial perturbations than would be expected of purely linear systems.

5. Producing an energy/frequency spectrum for each functionality of a material, molecule or collection of molecules develops the database. The difference between spectra for each difference in function is obtained using interferometry; in effect by subtracting one spectra from the other. The methods for developing the database are to use either a network analyzer, where an emission of electromagnetic energy at increasing frequencies is directed toward the target and the resulting signal, modified by its interaction with the target, is canned using an network analyzer or to use time domain principles where a signal is expanded using a fast Fourier transform and where the received signal is similarly expanded. The two results are then compared using interferometry.

6. From the database, specific frequencies can be identified where the difference in resulting amplitude between differing functions of the target is large. For diagnostic purposes,-an apparatus emitting a signal made up of the specified frequencies and receiving the resultant signal after exposure to the target enables a determination as to whether the energy absorption indicates the presence or absence of the suspect functionality.

7. In manufacturing applications, a similar system can be used to facilitate quality control and non-destructive testing. The frequency shift of a metal under tensile load can be determined. The frequency shift is an indication of the strength of the material and of its failure characteristics. That information can then be used by transmitting a signal at specific frequencies and measuring the resultant amplitude: comparing the actual result against the results obtained from the master sample indicates the quality and by extrapolation the strength characteristics of the metal.

8. Similarly, the change in resultant amplitude can be used to store or retrieve digital information. Many polymers change shape or conformation when exposed to high energy levels, whether that energy is provided by way of heat, e-m radiation or mechanically. The conformational change can be read remotely by using the apparatus. There are also likely to be applications within the fields of molecular [or quantum] computing where the invention provides a means of remotely reading and inputting data.

9. As a result of the penetrative nature of longer wavelength electromagnetic emissions, where a specific spectrum is known for a particular material, the invention can be utilized to identify the presence of alien materials being carried or included in material. For example, the present invention can be utilized to screen passengers boarding or leaving aircraft to detect the presence of contraband substances of any sort.

10. The apparatus consists of a signal generator, a transmitting antenna, a receiving antenna and a receiver circuit able to measure the amplitude of a received signal at specific frequencies. The frequency specification can conveniently be achieved using frequency filters.

11. The apparent conformation of a molecule or conjugation of molecules alters as the vibrational frequency of the target increases. That in turn alters the frequency shift. The combination of frequency shifts is particular to any single molecule or conjugation of molecules. As a result, the resultant amplitude [energy absorption] becomes a highly sensitive and accurate means of establishing molecular vibrational frequencies or temperature.

12. All of the above functions can be entirely non-invasive. At radio frequencies, low power (less than a milli-watt) electromagnetic radiation is deeply penetrative of most tissue and organic compounds. [Infrared light is penetrative of tissue to a depth of less than 2 mm whilst microwave frequency emissions are penetrative to a depth of up to 500 mm; longer wavelengths are fully penetrative]. Thus, a diagnostic test aimed at degenerative neuron diseases can be accomplished even where the affected tissue is deep within the brain of the patient. Similarly, if the temperature-measuring probe is used, the core temperature of a neo-natal infant can be established non-invasively. Comparison of animal core temperatures with skin temperatures is a significant measure of shock.

13. The underlying hypothesis postulates that conformational change is reversible. There is a very large body of evidence that this is so and general acceptance of this. [Change that involves altered conjugations of molecules is not necessarily reversible].

14. The perceived frequency shift provides an indication of the energy, expressed in amplitude and frequency, required to bring about the change. Therefore, by measuring the energy difference an indicative template of the energy required to reverse the change is obtained. That information can assist in identifying successful drugs or similar therapies.

15. The conformation of a molecule or conjugation of molecules can be described by a wave function equation. [Molecular wave function equations were pioneered and used by de Broglie and by Schrodinger; Feynmann/Wheeler derived wave function equations to describe complex and very large conjugations]. The difference between functionalities/conformations can also be described mathematically.

16. The basic constituents of a cell are known. What are unknown are the exact make-up of the cell and, particularly, the dynamic interactions of large molecules. Those dynamic interactions effect the conformation of the molecules and therefore their functions.

17. Three dimensional computational flow dynamics has been developed to allow very complex stream flow to be modeled. In particular, the development and behavior of vortices and their inter-action can be modeled.

18. By substituting the wave functions of the molecular constituents of a cell, or of a cell component, for the velocity vectors used in cfd, it is possible to construct a dynamic model of a cell, using cfd. The dynamic model is sensitive to changes. A change in DNA can be modeled in exactly the same way a change in a streambed can be modeled, while dynamic or functional changes can be modeled by altering the point change characteristics on the cell. The change in function resulting from an external change can be modeled and the effect of an external input (for example, a drug constituent) can also be modeled.

19. The principle constituents of a cell can be determined by flow cytometry and derived from published libraries. These constituents can be each described by their wave function or Schrodinger equation and these non-linear equations used in the construction of the model.

In summary, some of the practical emanations of the invention are:

Diagnostics, including very early stage or pre-symptom, that are non-invasive, in-vivo and that can be remote.

On line non-destructive testing and quality systems

Remote reading information systems

Remote reading and material specific temperature measurements

Drug templating and medicinal chemistry

Dynamic models of living organisms with applications in disease investigation, proteomics and in the development of drug and other therapies.

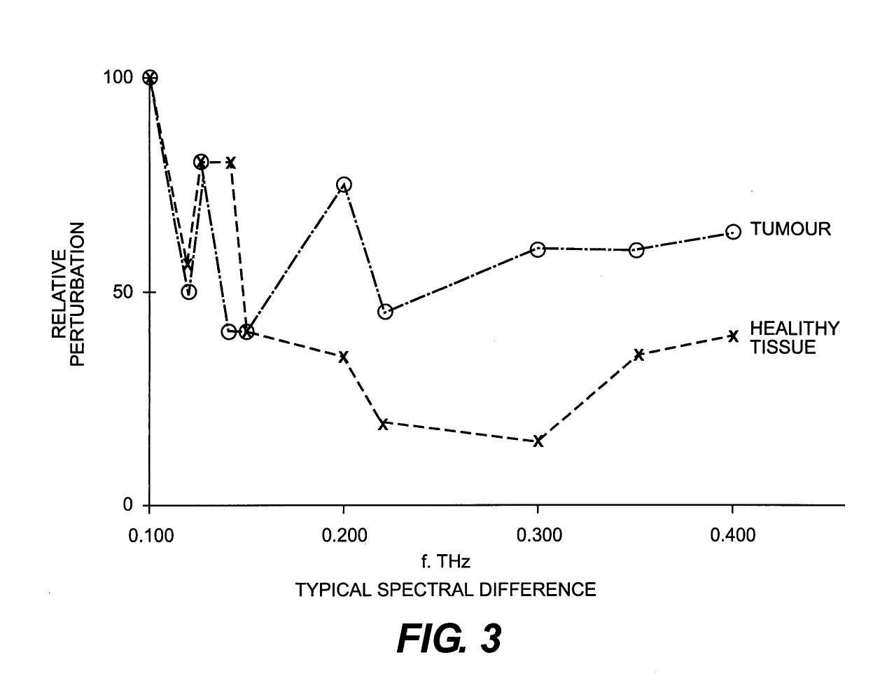

Examples of graphic information obtained using the methods of the present invention is provided in the accompanying drawing FIGS. 3-5.

As reflected in those drawings, in accordance with the present invention, the spectrum specific to an object or target can be found by exposing the target to a coherent waveform and recording the perturbation of that waveform, observed for example in FIG. 3. The higher the frequencies of the perturbed signal, the more specific to the individual constituent of the target the perturbed waveform is.

Thus the difference between two very dissimilar targets will be observable at a low frequency spectrum. Differences in the constituents of two closely related targets will be recorded as a higher frequency spectrum and differences in, for example, the conformation of otherwise identical molecules present in the targets will be recorded at very high frequencies. The unperturbed frequencies are present in the coherent transmitted signal: the amplitude of the harmonics reduces as the wavelength reduces and the frequency increases. The perturbation of the high frequency harmonics is then present in reducing amplitudes all the way down the frequencies of the received signal. In this way, the desired specific signal can be extracted by using Fourier transforms.

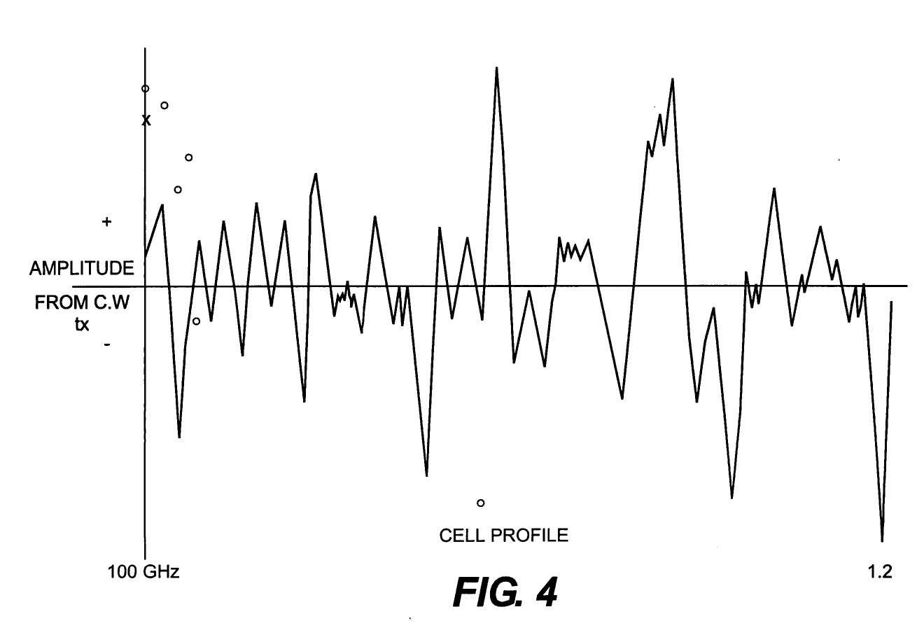

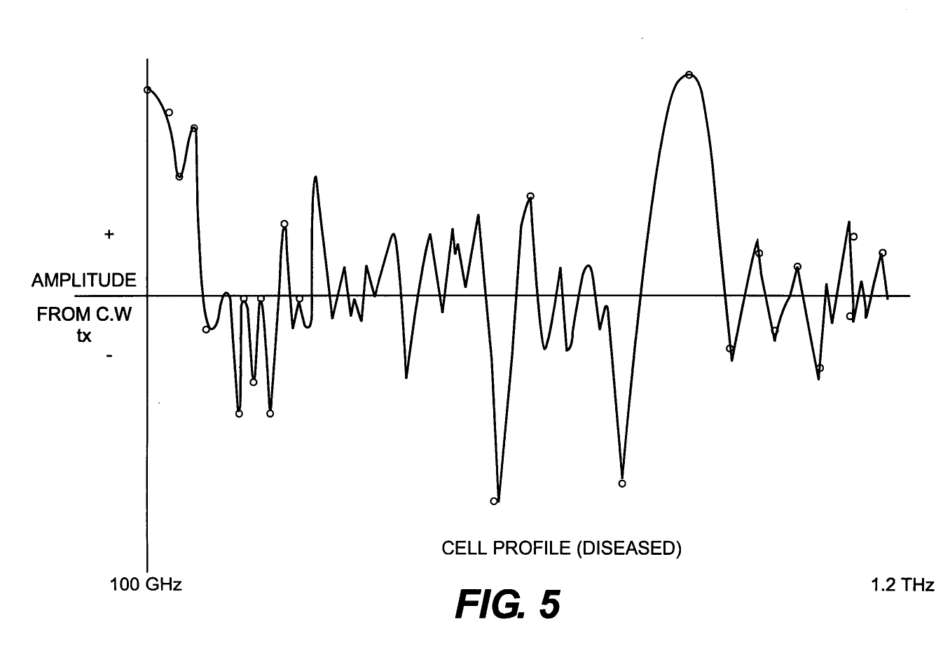

As shown in FIG. 4, the spectrum shown marked “cell profile” is the identifying lower frequency spectrum specific to a particular type of cell. As shown in FIG. 5, the spectrum shown marked “Cell Profile (diseased)” is the spectrum specific to the diseased version of the same cell type as that in the first spectrum. The graph as shown in FIG. 3 and identified as “Typical Spectral Difference” demonstrates the higher frequency spectra related to molecular differences between the diseased and healthy versions of a cell, where those molecular differences are the conformational variations of a specific large protein.

Additional embodiments of the present invention are as follows:

Industrial quality control can be achieved by non-destructive means. The function of the atoms and molecules that make up a material is effected by the form in which they exist. The differences in conformation are described as crystallinity, for example, in polymers and the degree and rate of polymerization is often characterized as being the degree of inter-molecular cross linking. This cross linking is related to the conformation of the polymer molecules.

The spectrum that is uniquely characteristic of the molecular or atomic conformation that is present in a material of preferred structural or material properties is determined by scanning a sample of the material that exhibits the standard properties. Thereafter, material is scanned to confirm that the specific spectrum is present. In this way, a non-destructive, on-line quality control can be exercised.

The spectrum of linear biological polymers is sensitive to small changes in those linear polymers. The specific spectral signature of individual DNA can be determined experimentally and thereafter that spectrum can be used for trace or identification purposes. The presence or absence of the specific spectrum is established by transmitting an appropriate principle frequency and determining whether the transformed received signal exhibits the perturbation and resulting spectrum of the known target.

The following U.S. patents and journal articles are incorporated herein by reference: U.S. Pat. No. 4,765,179; U.S. Pat. No. 6,287,874; U.S. Pat. No. 6,287,776; “An introduction to medical imaging with coherent terahertz frequency radiation:”, Fitzgerald et al., Phys Med Biol 47(7) R67-R84 (March 2002); Journal of Physics in Medicine and Biology: 47 #21; “THz time domain spectroscopy”, Markelz et al. Phys Med Biol 47 (2002) 3797-3805.

In short, the present invention provides the following features:

A means to identify the presence or absence of materials or of forms of material by utilizing a transmitted signal of the principal frequencies shifted from or to and comparing the changes in amplitude of the received signal with a known result.

An apparatus consisting of a means of generating a broad band width signal in wave lengths between 1,000,000 and 1 meters, an antenna directionally transmitting the signal, an antenna directionally receiving the signal, an amplifier and a means, typically a digital oscilloscope, of determining the amplitudes at the constituent frequencies of the signal, together with a means of calculating the higher frequency harmonics of the transmitted signal and a means of similarly calculating the harmonics of the received signal.

An apparatus consisting of a means of generating a number of signals of differing and specific wave lengths, a directional antenna, a directional receiving antenna, an amplifier able to amplify each signal and a means of comparing the amplitude of the received signals with a known pre-set spectrum of amplitudes versus frequencies where the higher range frequencies are synthesized using Fourier transforms.

A means to identify the presence or absence of organisms of differing function by utilizing a transmitted signal of the principal frequencies shifted from or to and comparing the changes in amplitude of the received signal with a known result where the higher range frequencies are synthesized using Fourier transforms A means to identify the presence of altered function in living organisms by determining amplitude changes at specific frequencies within the sub-microwave spectra following interaction with the object.

A means to obtain an indication of the amount and form of electromagnetic energy required to bring about a change ire conformation (alternatively electromagnetic geometry) of a molecule or conjugation or arrangement of molecules A means to obtain a description of the amount and form of electromagnetic energy required to reverse a change in conformation observed by the invention A means to produce a dynamic three dimensional model of cell structure and components based on three dimensional flow theory wherein the major entities are represented by vortices.

A means to characterize the individual components of a living organism at cellular level by simulating the energy flow and thus describing the entities present in the cell by means of wave function or other non-linear equations.

A means to simulate the functional changes in a living organism at cell level or below caused by changes in the conformation of the entities.

A means to simulate the effects of outside stimuli at an electromagnetic level on a living organism at cellular level or below and by such stimulation to simulate the functional effects of such stimuli.

A means to simulate the effects of outside stimuli characterized as therapeutic drugs on the functionality and conformations of a living organism at cell level.

A means to simulate cell dysfunction and by such simulation to isolate the early stage changes that result in identifiable dysfunction.

A means to determine the presence or absence of the individual electromagnetic spectra that are specific to particular conformations of atomic and molecular entities, using coherent or continuous wave signals within the radio spectrum at wave lengths that can permit in-vivo examination and to identify the spectrum by observing the perturbation of higher frequency harmonics (up to and including the tera hertz frequencies) of the transmitted signal, using Fourier transforms of both the transmitted and received signal following interaction with the object.

It is thus submitted that the foregoing embodiments are only illustrative of the claimed invention and not limiting of the invention in any way, and alternative embodiments that would be obvious to one skilled in the art not specifically set forth above also fall within the scope of the claims.

The following examples are presented as illustrative of the present invention or methods of carrying out the invention, and is not provided as restrictive or limiting of the scope of the invention in any manner.

### EXAMPLES

### Example 1

Prion Testing and Detection of Specific Molecules

A method is described whereby the presence or absence of a specific conformation of a protein can be identified in a living organism or animal, including a human, rapidly and non-invasively. An example is the prion protein associated with the transmittable spongiform diseases of the brain, for instance BSE in cattle: the PrPsc form of the prion protein is a different conformation of the normal prion protein produced by neurons.

The identifying spectrum for normal prion protein and for the unusual conformation prion proteins is first established by exposing samples of each material to a coherent wave signal as described herein and determining the perturbed signal resulting from the interaction of the transmitted signal with the target material. That spectrum can be determined irrespective of whether the target material is in solution with other materials.

For mass in-vivo examination, an apparatus consists of the method of producing a coherent principal signal as described herein together with the receiver and pre-amplifier described. A detector circuit can be used to determine if the specific spectrum associated with the target material is present. By scanning the candidate the presence or absence of that specific spectrum is found. Such a scan is very rapid: provided that a number of pulses of the transmitted signal are made the probability of the target molecules being absent or present, depending on whether the specific spectrum is observed, can be very high. The scanning device can most conveniently be a simple probe, with a head containing both transmitter and receiver antenna. The head can be passed closely over the target area.

The method is intended primarily for the detection of conformational differences in proteins present normally where those conformational differences result in cell degradation diseases. It is anticipated that the method will prove to be specifically useful in the identification of early stage protein changes related to for instance cancer as well as the degenerative diseases of neurons described above.

The method can also determine the presence or absence of specific molecules in small quantities and is useful therefore in determining whether narcotics are present, or to determine the presence of metabolites specifically associated with cardiac disease.

### Example 2

Creation of Drug Templates to Correct Cell Abnormality

Where a specific spectrum is detected associated with the conformational abnormality of a protein linked to a disease, the method is used in the templating of drug candidates for therapies intended to correct the conformational abnormality. The spectral difference observed as described herein can be quantified in terms of frequency and amplitude: that is done by subtracting the spectrum of the normal conformation from that of the abnormal. The difference between the two, expressed in frequency and amplitude, is the energetic characteristic difference (ECD). The ECD is net of the energy required to initiate the change (to overcome the inertia of the normal conformation). The ECD can be written as a non-linear wave equation that describes the difference: the opposite of the ECD (that is, by substituting negatives for positives and vice versa) is an indication of the energy, net of the energy required to initiate change, required to reverse the change. The system thereby provides a useful indication of candidate drug compounds or therapies that can result in the conformational change being reversed and the resulting cell abnormality being corrected.

### Example 3

Cell Sorting Device

The changing characteristics of sperm cells as they mature appears to indicate that initially male and female sperm cells are at different points in their functional change and that functional change is characterized by alterations in the ion transfer rates. As a result, the invention allows the proportion of male to female cells to be determined and the cells can then be divided into male and female groups.

In another specific embodiment of the invention, a cell sorter is provided which makes use of the method of the invention. A method to sort cells by bulk iterative methods. Where cells of two or more types are mixed together, such as in the ejaculate of a male mammal, the present invention can be utilized to sort the cells. The spectral difference between, for example, a male cell and a female cell is established. A sample already sorted into cells of one type is scanned by passing a coherent wave through the sample and recording the perturbed signal. That spectrum is compared to a similar signal obtained by scanning the cells of the second type. Comparing the two spectra the critical differences within the spectra, in terms of frequencies are established. One or more specific principal frequencies can be selected: in the case of cells of one sort, the perturbation results in a significant frequency shift from a principal frequency: that shift is shown as a decrease in the amplitude of the transmitted frequency and an increase in another, adjacent frequency. In the case of the cells of the second type, a second principal frequency with its commensurate shifts is selected.

Where a mixture containing quantities of the two types of cell is scanned with a coherent signal containing the two principal frequencies described, the relative amplitudes of the shifts can be measured. By applying the rule of mixtures, the relative amplitudes indicate the proportions of the two cells, each to the other.

An example is described below:

The machine is intended to sort animal semen into male and female, with a high probability (in excess of 93%) and a residue of indeterminate probability.

The method of operation is to load the machine with untreated ejaculate, through the load funnel. This produces nine equal portions, each of which is held in a determinant area.

Each determinate area is faced with an RF transmitter and backed by an RF receiver.

When the area is subjected to a pulse of specific wavelength, the absorption of energy is measured by comparing the transmitted energy, less the known normal loss of the system without ejaculate, with the received energy.

The absorption at that frequency of male cells and of female cells is known and by applying the rule of mixtures the proportions of male and female cells is known.

Samples that are predominantly male (60% or higher) are then sorted to one side and redivided. Samples that are predominantly female are sorted to the other side and redivided. The process is then repeated.

Where a sample exhibits a high proportion of one sex or the other it is moved toward the end of the sort process and stored there until further samples of a similar concentration are derived. The methods of moving the ejaculate mimic the natural peristaltic action of ejaculation. The sample is kept between two, thin, flexible membranes.

A layer of magneto-rheological fluid that increases steeply in viscosity and density when a magnetic field is applied backs each membrane. By applying localized magnetic fields to the fluid, the space between the membranes can be minimized and the sample moved or stored at will.

Components.

The cell sorting machine used in the process described above has The machine consists of the following main components.

Outer casing

Clamping mechanism

Entry funnel

Membrane glove

Magneto-rheological fluid

Encapsulated sensors/fields

Exit funnel

Electronic control unit.

Construction of the device in accordance with the invention is as follows:

A. The outer casing is manufactured in two, opposing halves. It is made from thermo-formed plastics (typically, ABS) although if desired it can be gravity die cast in aluminum. The benefits of ABS moulding are reduced part price, reduced tooling cost, reduced time to production and good but limited protection from impact damage. The two halves locate to each other using dowel pins with butterfly nuts at each end.

The dowel pins locate in sleeves hot-staked into the moulding. (If an aluminum casting is used, the sleeve is a drill and ream operation. The casing is molded with vents to allow the electronics to be cooled and incorporates a single DIN socket on one half and two DIN sockets on the other. One DIN socket carries the ribbon cable connector between the units. The second is the power supply and data link. The casings are provided with a means to be stand alone units but also have a carrying strap connection that allows the unit to be hung on, for example, a stand.

B. The clamping device fits over the exterior halves at the top and positions the casing correctly. The membrane glove is drawn up though the center slot and clamped in position by:

C. The dividing funnel. In operation the ejaculate is poured into the funnel and the division divide the ejaculate in the nine samples.

D. The membrane glove is, in effect, one glove sealed inside another, with a flange around the edge that is trapped within the outer casing. The space between the inner and outer glove is filled with magneto-rheological fluid (such as that manufactured by Lord Industries). The glove is made from soft nitrile or latex material and the inner and outer are heat staked together after filling with the m-r fluid to contain the fluid evenly in the interstice. The glove is also heat staked so that it is divided into vertical sections in the antenna areas.

E. Magneto-rheological fluid is manufactured by Lord industries. It is now being used in adaptive damping systems for General Motors and is therefore widely and economically available. When excited by a local magnetic field, the fluid becomes very viscous (close to solid) and as a result the localized volumes increase. The response rate for the change is in the order of milli-seconds.

F. Important elements of the device are the antennae and the electromagnets that generate the magnetic fields. The antennas are pyramidical in shape and are formed by vacuum forming a polyester (Mylar) film of a thickness of between 100 and 150 micron. The vacuum formed pyramids shapes are part of a larger vacuum forming that encompasses 3 mm diameter hemispherical slots arranged at 0 degrees and 90 degrees to the X axis of the machine. Each slot can carry an electromagnet that consists of a fine wire winding over a polymer core. The outward facing (that is, facing towards the sample) face of the Mylar vacuum forming is metallized. A simple vacuum formed pyramid is metallized with a ‘bow tie’ dipole at 0 degrees to X, with a second identical part metallized at 90 degrees to X. One such forming is inserted in the main pyramid to form respectively the transmitter and the receiver. The dipole has a wire termination that is inserted through small holes in the main pyramid; these holes are formed during the vacuum forming process. In this way, the spacing between the reflector and the dipole is maintained. [The technology is NASA developed but is freely available.] Following assembly and insertion of the electromagnets the entire assembly is encapsulated using a standard potting compound, to provide a smooth and flat surface against which the glove is stretched.

G. The exit funnel for the glove allows for three drains as described and stretches the base of the glove into position.

H. The electronic controls consist of a printed circuit board that backs the encapsulated parts (F). The circuit board provides discrete connections to each transmitter and to each electromagnet. The system is driven using standard algorithms with the e.c.u. On initiation, a series of 100 pulses is transmitted from each of the first nine antennas in turn and the received signal analyzed. The received signal is compared against the received control signal that is established by transmitting with no ejaculate in the system. The result is then compared against the established absorption or perturbation levels for male and female cells. The absorption or perturbation levels indicate the proportion of male to female cells. At that point, each antenna pocket is given a value derived from the male: female absorption or perturbation. The ejaculate is confined laterally by the heat staking and vertically because the electromagnet at the base of the antenna pocket is activated, so that the expanded magneto-rheological fluid forms a barrier. The samples are now moved vertically and laterally using the magneto rheological effect so that nine new division can be created graded as to the highest male in the one or two left hand antenna pockets, the highest female in the one or two right hand antenna pockets and the balance equally redivided across the center pockets. The process continues until at least three antenna pockets on the left have an acceptably high male proportion and the three on the right have an acceptably high female proportion. Because the sample can be moved vertically up and down between antenna rows, the first stage sort/measure process can be repeated until an improvement over the first sort has been obtained.

Software

The information aspect of the sorting device consists of a pulsed transmitter and a filtered receiver. The machine can be supplied with alternate frequencies so that the same device can be used for different species semen or alternatively to predominate male or female cells. The receiver filter can also be set to different values. Since the machine is intended to self calibrate (that is, the background noise and absorptions or perturbations are zeroed by cycling the machine dry, without ejaculate) these adjustments can be carried out within software by pulsing consecutively at rising frequencies and choosing only to analyze the received pulses in a sequence that relates to the sample. In other words, where the machine is set to be able to handle five different species, it will pulse a sequence of five signals. The receiver will only read every fifth signal, commencing with the one appropriate for the sample under consideration. It is intended that a complete sort record will also be maintained and it is possible that the final stage of each sort can give some degree of quality control by being programmed to consider a protein conformation.

What is claimed is:

1. A method for detecting an abnormal condition in nucleic acids, proteins, cells, tissues or organisms comprising:
a. applying a signal of electromagnetic radiation generated as a series of harmonics to a normal or standard nucleic acid, protein, cell, tissue or organism so as to obtain a normal or standard spectrographic waveform pattern of the absorbance or perturbance of the radiation by the normal or standard object;
b. applying the same signal to a target nucleic acid, protein, cell, tissue or organism suspected of having an abnormality so as to obtain a spectrographic waveform pattern of the absorbance or perturbance of the radiation by the target object; and
c. comparing the spectrographic waveform pattern of the target object with the spectrographic waveform pattern of the normal or standard object to determine the presence or absence of an abnormal condition.

2. The method according to claim 1 wherein the applied signal of electromagnetic radiation is in the tera hertz range.

3. The method according to claim 1 wherein the applied signal of electromagnetic radiation is a broad band signal with wave lengths between 1,000,000 and 1 meters.

4. The method according to claim 1 wherein the target object is a neuron.

5. The method according to claim 1 wherein the target object is selected from the group consisting of an ameyloid precursor protein, a tau protein and a prion protein.

6. The method according to claim 1 wherein the spectrographic waveform pattern is obtained using Fourier transforms.

7. An apparatus capable of carrying out the method of claim 1 comprising:
a. a means of generating an electromagnetic signal having a wave length between 1,000,000 and 1 meters;
b. an antenna for directionally transmitting the signal and an antenna for directionally receiving the signal;
c. an amplifier and a means of determining the amplitudes at the constituent frequencies of the signal;
d. a means of calculating the higher frequency harmonics of the transmitted signal; and
e. a means of similarly calculating the harmonics of the received signal.

8. The apparatus according to claim 7 wherein the means to determine the amplitudes at the constituent frequencies of the signal comprises an oscilloscope.

9. A method for differentiating and separating male and female sperm cells comprising:
a. obtaining an animal ejaculate and dividing the ejaculate into portions to be scanned with a signal of electromagnetic radiation generated as a series of harmonics;
b. scanning said portions of the ejaculate with a signal of electromagnetic radiation generated as a series of harmonics and determining the percentage of male and female sperm cells in the scanned portion by comparing the spectrographic pattern of the absorbance or perturbance of the signal in the scanned portion with the known spectrographic pattern made from an ejaculate having equal numbers of male and female sperm cells;
c. separating those portions which have a predominant amount of male or female sperm cells; and
d. repeating the above steps and collecting the ejaculate portions containing sperm cells of predominantly one sex until a desired percentage of sperm cells of that sex is obtained.

10. An apparatus capable of carrying out the method of claim 9 comprising:
a) a means of generating and receiving broad band width electromagnetic signal in wave lengths between 1,000,000 and 1 meters;
b) an antenna for directionally transmitting the signal and an antenna for directionally receiving the signal;
c) a means for receiving and portioning an animal ejaculate;
d) a means of calculating the absorption or perturbation pattern of electromagnetic signals after said signals are directed to portions of the ejaculate so as to determine if said portions contain a predominant percentage of sperm cells of one sex; and
e) means for collecting portions of the ejaculate which are identified as containing sperm cells which are predominantly one sex at a desired percentage.

11. A method of determining the presence or absence of a particular conformations of atomic and molecular structures comprising scanning said structures with a signal of electromagnetic radiation at wave lengths that can permit in-vivo examination, and identifying said conformations by observing the absorption or perturbation of higher frequency harmonics up to and including the tera hertz frequencies of the transmitted signal using Fourier transforms of both the transmitted and received signal following scanning of the structures.

12. A method of diagnosing a tumorous condition in a patient comprising:
a. applying a signal of electromagnetic radiation to a sample known to reflect a tumorous condition so as to establish a standard spectrographic waveform pattern of the absorbance or perturbance of the electromagnetic radiation of the known sample indicative of a tumorous condition;
b. applying the same signal to a target tissue suspected of having a tumorous condition so as to obtain a spectrographic waveform pattern of the absorbance or perturbance of the electromagnetic radiation in the tissue to be tested; and
c. comparing the spectrographic waveform pattern of the tested tissue with the pattern given by the known tumorous pattern to determine the presence of absence of the tumorous condition.

13. An apparatus capable of performing the method of claim 12 comprising a means of generating a number of signals of differing and specific wave lengths, a directional,antenna, a directional receiving antenna, an amplifier able to amplify each signal and a means of comparing the amplitude of the received signals with a known pre-set spectrum of amplitudes versus frequencies.

14. A method of identifying the presence or absence of substances in an object to be tested comprising transmitting an electromagnetic signal through a control object having known substances so as to create a standard absorption or perturbation spectrum for the object at a given wavelength, scanning the test object using the same electromagnetic signal so as to obtain an absorption or perturbation spectrum for the object to be tested, and comparing the changes in amplitude of the spectral pattern in the tested object so as to determine the presence or absence of substances as compared to the standard object.

15. A method of identifying the presence or absence of organisms of differing function comprising transmitting an electromagnetic signal of the principal frequencies through a sample suspected of containing such organisms and comparing the changes in amplitude of the received signal with a known result to determine the presence or absence of the desired organisms.

16. The method according to claim 15 wherein the higher range frequencies are synthesized using Fourier transforms.

17. A method of identifying the presence of an altered function in living organisms comprising transmitting an electromagnetic signal generated as a series of harmonics through a living organism suspected of having an altered function and determining amplitude changes at specific frequencies within the sub-microwave spectra following interaction with the organism.

18. A method of producing a dynamic three dimensional model of cell structure and components comprising applying a signal of electromagnetic radiation generated as a series of harmonics to said cell, receiving said signal, and transforming said signal using a Fourier transformer so as to obtain a spectrographic waveform pattern of the electromagnetic radiation which reflects a dynamic three dimensional model of the cell structure and components wherein the major components are represented by vortices.

19. The method according to claim 18 wherein the components present in the cell by identified means of wave function or other non-linear equation.

20. A method of drug templating of candidates for particular drug therapies comprising identifying a specific spectrum of a protein associated With a conformational abnormality linked with a disease by scanning said protein with electromagnetic radiation generated as a series of harmonics so as to obtain a spectrographic waveform pattern of the absorbance or perturbance of the radiation caused by said conformational abnormality, introducing a drug suspected of treating the disease to the abnormal protein and obtaining an electromagnetic scan of the protein after interaction with the drug, and determining the efficacy of said drug by comparing the conformation of the protein following treatment to determine if the conformational abnormality has been reversed in whole or part.
